(19) United States
(12) Patent Application Publication
(10) Pub. No.: US 2005/0069588 A1
(43) Pub. Date: Mar. 31, 2005

(54) Preparation containing polydimethylsiloxane for nail, cartilage,bone, joint,muscle and tendon disorders

(76) Inventor: Leendert Taal, Veldhoek/Hengelo Gld (NL)

Correspondence Address: YOUNG & THOMPSON, 745 SOUTH 23RD STREET, 2ND FLOOR, ARLINGTON, VA, 22202, US

(21) Appl. No.: 10/473,968

(22) PCT Filed: Mar. 4, 2002
(86) PCT No.: PCT/NL02/00143

(30) Foreign Application Priority Data
Mar. 2, 2001 (NL) 1017487

(51) Int. Cl. A61K 35/78; A61K 33/32; A61K 31/7008; A61K 31/4188; A61K 31/198

(52) U.S. Cl. 424/486; 424/769; 514/62; 514/393; 514/562; 514/564; 424/642

(57) ABSTRACT

The present invention relates to a topical preparation that contains polydimethylsiloxane for the external treatment of nail, cartilage, bone, joint, muscle and tendon disorders. The preparation can also contain biotin, zinc, lysine, methionine, laurel extract, carotene, casein, collagen, glucose, a glucosamine and/or arnica extract. In particular the invention relates to a combination therapy where a glucosamine is administered orally.

The present invention relates to a preparation for the treatment of nail, cartilage, bone, joint, muscle and tendon disorders. In particular, the invention relates to a preparation for the treatment of lower legs, feet and hooves of horses.

The treatment of nail, cartilage, bone, joint, muscle and tendon disorders is generally carried out by combating the disorder internally or externally at the site of the disorder. In WO 00/15202, for example, it is described that nail disorders can be treated by topical application of an agent that contains an active ingredient in combination with a carrier based on a C₁-C₄ alkyl ester of lactic acid, malic acid, tartaric acid or citric acid. Antibiotics and disinfectants can, for example, be incorporated as active ingredients.

U.S. Pat. No. 4,996,043 describes a composition for the treatment of keratinous material, in particular horses' hooves, which contains an aqueous dispersion containing 20-90% water, oils with a high content of mono-unsaturated fatty acids and water-soluble or -swellable polysaccharides. Furthermore, a maximum of 10% silicone oil can be added.

The disadvantage of the preparations according to the state of the art is that they do not provide adequate transfer of the active component to the surface to be treated. As a result the active component does not penetrate adequately. Furthermore, in particular in the case of the treatment of horses, it is important that the preparation remains on the location to be treated for some considerable time and is not substantially removed by, for example, walking in pasture.

It has now been found that nail, cartilage, bone, joint, muscle and tendon disorders can be effectively prevented and/or treated by the use of a preparation that contains an active ingredient and, as carrier, at least 50% (m/m), based on the weight of the preparation, of polydimethylsiloxane. Polydimethylsiloxane, which is also termed dimethicone or dimethylsilicone, is a compound having the following structural formula:

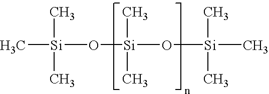

It is known as an agent for the protection of the skin, for example in the case of decubitus, incontinence and dermatoses. It has now been found that polydimethylsiloxane is an excellent carrier in the case of the treatment of the abovementioned disorders. In particular this agent increases the permeability of the tissue to which it is applied, as a result of which the action of active components that are present in the agent is increased. As illustrated in the examples, experiments with 30 horses suffering from diverse tendon and hoof disorders have shown, according to the protocol, that an appreciable improvement in the condition of the horses was obtained using the preparation according to the claims.

Preferably, a polydimethylsiloxane having a viscosity of 10,000 to 100,000 cSt (mm²/s), preferably 40,000 to 80,000 cSt, is used. The preparation preferably contains at least 70% (m/m) polydimethylsiloxane. Furthermore, the preparation is essentially anhydrous, that is to say it contains less than 20% (m/m), preferably less than 10% (m/m), and most preferentially less than 5% (m/m) water. It is also possible to use an activated polydimethylsiloxane, for example activated with silicon dioxide (simethicone). The percentage of silicon dioxide can then be approximately 2 to approximately 20% (m/m), based on the total preparation.

The active ingredients incorporated in the preparation of the invention are preferably one or more of the following: biotin, zinc, lysine and methionine, preferably a combination of all these substances.

Biotin (hexahydro-2-oxo-1H-thienol[3,4-d]imidazole-4-pentanoic acid) is a growth factor which is present in small amounts in every cell. The quantity of biotin in the preparation according to the invention is 10 to 100 gram per 400 gram polydimethylsiloxane. Biotin is a basic raw material for the production of keratin (the hoof in horses and the nails in man).

Furthermore, stress is reduced, as a result of which reconditioning proceeds more efficiently. Stress has the effect of destroying the growth process. Lysine and methionine are essential amino acids. The preparation according to the invention contains 1 to 20 gram lysine and 1 to 20 gram methionine per 400 gram polydimethylsiloxane. Together with biotin, lysine has an effect on the formation of keratin. Together with methionine, lysine forms keratin from the epithelium. Zinc is present in the preparation in an amount of 1 to 10 gram zinc per 400 gram polydimethylsiloxane. Zinc acts as a catalyst which in combination with biotin, lysine and methionine forms and produces keratin in the epithelium under the influence of oxygen-rich blood.

In addition to the abovementioned active ingredients, one or more of the following can also be added to the preparation: laurel extract, carotene, caseinate, collagen, glucose and arnica extract.

Laurel extract is obtained from the laurel berry, Laurus nobilis L. The laurel extract can be in the form of a laurel powder, but can also be a laurel oil. The laurel extract is obtained in the manner known in the art. The preparation according to the invention contains 1 to 30 gram of laurel extract per 400 gram polydimethylsiloxane. Laurel extract promotes the growth maturing from keratin. It also prevents premature aging of tissue, for example in horses in the further outgrowth region of the hoof, it guarantees an improvement in the elasticity of the horn wall.

Carotene, in particular β-carotene, is also referred to as provitamin A. It occurs in, for example, carrots and palm oil. In horses, for example, carotene indirectly and directly produces improved keratin production in the growth region of the hoof, at the transition from the leathery skin to the hoof horn. The preparation according to the invention contains 1 to 20 gram carotene per 400 gram polydimethylsiloxane.

Caseinate or casein is a mixture of phosphorus proteins and has a high content of essential amino acids. It can be obtained from milk in the conventional manner. Preferably (calcium) caseinate from cow's milk is used. The preparation contains 1 to. 20 gram caseinate per 400 gram polydimethylsiloxane.

Collagen is a polypeptide which comprises one third of the total protein in vertebrate mammals. It is one of the most important constituents of the skin, tissue and the organic component in bones and teeth. The preparation contains 5 to 30 gram collagen per 400 gram polydimethylsiloxane.

The topical preparation preferably also contains a glucosamine, derivatives thereof and/or salts, which gives particularly favourable results. Preferably an acetylglucosamine is used and more preferentially N-acetyl-D-glucosamine or a pharmaceutically acceptable salt thereof, in particular N-acetyl-D-glucosamine HCl.

An alternative component that can be incorporated in the topical preparation is arnica extract. This is obtained from Arnica montana L. Arnica oil is preferably used in the preparation according to the invention. The quantity of arnica extract is 1 to 10 gram per 400 gram polydimethylsiloxane. It reduces pain in the case of injuries and improves the recovery process.

According to another aspect of the invention three separate topical preparations are provided. The first preparation comprises polydimethylsiloxane and one or more, preferably all, of the following: biotin, lysine, methionine, zinc, caseinate, carotene, laurel powder and glucosamine. This first preparation is in particular suitable for the treatment of nails, hooves, cartilage and joints. The second preparation comprises polydimethylsiloxane and one or more, preferably all, of the following: glucose, collagen, carotene, caseinate, laurel powder and glucosamine. This second preparation is in particular suitable for the treatment of tendons and muscles. The third preparation contains polydimethylsiloxane and arnica extract. This third preparation is in particular suitable for pain control.

The nail, cartilage, bone, joint, muscle and tendon disorders which can be treated using the preparation according to the invention can exist both in humans and in animals. Examples of disorders in humans are rheumatic disorders such as Cushing's syndrome, osteoporosis, rheumatoid arthritis and arthrosis, cartilage degeneration, necrosis, joint disorders, joint mobilisation and impaired growth. The topical agent is applied to the intended location at least once a week. In addition it has been found that the preparation can advantageously be used in the case of tendon and ligament disorders.

It has been found that the preparation according to the invention is particularly suitable for the treatment of disorders in horses, in particular disorders of the hooves, feet and lower legs, most preferentially of hooves.

Examples of disorders which can be treated using the preparation according to the invention are:
loose wall, an interruption in the union between the supporting rim of the horn wall and the horn sole in the white line or in some other location;
hollow wall, detachment between various laminae of the wall;
horny pillar, a cylindrical renewal of the horn in the longitudinal direction on the inside of the horny wall;
horn cracks, to be differentiated according to location, extent and depth. They mostly occur on the horn wall. The horn cracks in the wall run in the longitudinal direction, that is to say in the direction of the horn bars. There are said to be bearing surface cracks, coronet cracks and continuous cracks, from the coronet to the bearing surface;
navicular disease, inflammation of the navicular bone, the collective name for frog, mucus bursa and coffin bone flexor tendon. Chronic inflammation of this area is termed chronic navicular disease (Podotrochleitis chronica);
crumbly hoof;
hard hooves;
red frog;
cancer of the frog; and
hoof cancer.

In addition to the topical application of the abovementioned preparation based on polydimethylsiloxane, it has been found that combination with an oral preparation that contains a glucosamine, derivatives thereof and/or salts gives particularly favourable results. Preferably, an acetylglucosamine, more preferentially N-acetyl-D-glucosamine or a pharmaceutically acceptable salt thereof, in particular N-acetyl-D-glucosamine HCl, is used. The quantity of glucosamine per day is 100 to 2,500 mg, preferably 1,000 to 2,000 mg.

The combination of the topical preparation with a glucosamine is in particular suitable when improvement in the cartilage and protection and build up of the joint is desired. In addition this combination can be used to improve the function or reconditioning of tendons and muscles.

The oral preparation that contains a glucosamine can be, for example, a capsule, tablet, powder or drink solution. It contains a carrier suitable for the type of dosage form. In particular, the oral preparation that contains a glucosamine is a tablet with, as carrier, one or more of the following: gelatine, magnesium stearate, sugar and lactose.

The topical preparation according to the preparation can have, for example, the following compositions:

Preparation 1
(in particular suitable for the treatment of nails,
cartilage, bones and hooves)

| | |
|---|---|
| polydimethylsiloxane (dimethicone, viscosity 60,000 cSt) | 400 g |
| biotin | 60 g |
| lysine | 7.5 g |
| methionine | 7.5 g |
| zinc | 2.3 g |
| caseinate | 4 g |
| carotene | 2 g |
| laurel powder | 8 g |
| N-acetyl-D-glucosamine HCl | 1.5 g |

Preparation 2
(in particular suitable for treatment of joints,
muscles and tendons)

| | |
|---|---|
| polydimethylsiloxane (dimethicone, viscosity 60,000 cSt) | 400 g |
| glucose syrup | 40 g |
| collagen | 20 g |
| carotene | 10 g |
| caseinate | 10 g |
| laurel powder | 10 g |
| N-acetyl-D-glucosamine HCl | 1.5 g |

Preparation 3

| | |
|---|---|
| polydimethylsiloxane (dimethicone, viscosity 60,000 cSt) | 400 g |
| arnica oil | 6.5 g |

When treating with the topical preparation the latter is applied to the site of the disorder. Fresh preparation is applied regularly, for example once a week. The treatment continues as long as is necessary to heal the disorder. In general this will be a period of 1 to 6 months.

The oral preparation containing glucosamine is preferably administered in combination with the topical preparation. The oral preparation is administered daily, for example in a dosage of 1 gram per day. Optionally this daily dose can be split into several doses.

### EXPERIMENTAL

Various horses were treated in accordance with the protocol for 6 months with the topical and optionally the oral preparation according to the invention. An appreciable improvement in the condition of the animal that was treated was found to take place. A number of cases are described in detail below.

Case 1 Hoof Cracks—Bearing Surface Crack

Horse with aging hoof cracks caused by poor elasticity and by poor keratin production by the hoof horn. Loading stress too great for the poor horn quality. Clinical picture: uncomfortable gait.

Treatment

Smear hoof ointment (Preparation 1) into coronet once a week. 1 gram per day N-acetyl-D-glucosamine HCl, administered orally.

As a result of treatment, development of a good horn structure is taking place in the outgrowth region and the horn elasticity is improving. The structural change from the outgrowth region to the dying-off region proceeds more regularly. As a result of the above changes the forces can be relaxed in such a way that the crack does not again continue into the transition region. The aging crack is starting to disappear. The stress indication line is disappearing. Clinical picture: horse moves comfortably.

Treatment

Smear hoof ointment (Preparation 1) into coronet once per week. Oral administration of powder stopped.

As a result of further treatment, good horn structure with more than adequate elasticity. The dying-off region has become small. The stress and aging crack is restricted to the dying-off region. Clinical picture: horse moves comfortably and tactmatically.

Case 2 Hoof Cracks—Coronet Crack

The horse has developed hoof cracks as a result of overstressing in combination with poor elasticity of the keratin union of the hoof horn and poor quality of the cartilage in the hoof, resulting in a hoof mechanism that works poorly. The loading stress is too great, as a result of which the hoof splits open from the coronet. Clinical picture: walks gingerly.

Start of Treatment

Smear hoof ointment (Preparation 1) into coronet once a week. 1 gram per day N-acetyl-D-glucosamine HCl, administered orally.

As a result of treatment the coronet is becoming more supple and the horn elasticity has increased, crack is no longer open and is gradually closing by growth from the inside. Clinical picture: horse walks comfortably and tactmatically.

Treatment

Smear hoof ointment (Preparation 1) into the coronet once per week.

As a result of further treatment the coronet has become supple. Horn elasticity is good, crack has closed up. Very good recovery of the coronet. Clinical picture: horse walks comfortably and tactmatically.

In a corresponding manner, the use of the hoof ointment (Preparation 1), on its own or in combination with the oral preparation, was found to be suitable for the treatment of loose wall, tilting of the coffin bone, horny pillar, seizure of the hoof mechanism and ball arthrosis.

Case 3 Tearing of the Flexor Tendon

The flexor tendon of the right hind leg of the horse was ruptured. Immediately after the tendon was ruptured it was established by means of a tendon scan that only 10% of the tendon was still attached, with a large amount of scar tissue and blood clotting. Clinical picture: the leg was very swollen and no load was put on it.

Treatment: Smearing with tendon ointment (Preparation 2) and 1 gram per day N-acetyl-D-glucosamine HCl, administered orally.

As a result of the treatment, during the first month the horse cautiously started to put weight on the leg and its ball gave way to a considerable extent (almost down to the ground). The swelling gradually decreased. During the second and third months the horse started to put increasing weight on the leg. The ball also gradually gave way to a lesser extent and the swelling in the leg disappeared. The horse started to put weight on the leg when at rest. A further tendon scan was made after the third month. The tendon was 75% recovered without visible scar or connective tissue.

1. Topical preparation that contains an active ingredient and, as carrier, at least 50% (m/m), based on the weight of the preparation, of polydimethylsiloxane for the external treatment of nail, cartilage, bone, joint, muscle and tendon disorders.

2. Preparation according to claim 1, wherein the polydimethylsiloxane has a viscosity of 10,000 to 100,000 cSt.

3. Preparation according to claim 1, wherein the active ingredient is chosen from biotin, zinc, lysine, methionine and mixtures thereof.

4. Preparation according to claim 3, that contains biotin, zinc, lysine and methionine.

5. Preparation according to claim 1, that further contains a glucosamine, derivatives thereof and/or salts thereof, preferably N-acetyl-D-glucosamine or a pharmaceutically acceptable salt thereof, more preferentially N-acetyl-D-glucosamine HCl.

6. Preparation according to claim 1 that further contains one or more of the following: laurel extract, carotene, casein, collagen, glucose and arnica extract.

7. Preparation according to claim 1, that contains biotin, lysine, methionine, zinc, casein, carotene, arnica extract, laurel extract and N-acetyl-D-glucosamine HCl.

8. Preparation according to claim 1, that contains glucose, collagen, carotene, casein, laurel extract and N-acetyl-D-glucosamine HCl.

9. Preparation according to claim 1, that contains arnica extract.

10. Preparation according to claim 1 for the treatment of horses.

11. Preparation according to claim 10 for the treatment of disorders of the hooves, feet and lower legs of horses.

12. Preparation according to claim 11, for the treatment of horses' hooves.

13. Preparation according to claim 1 for the treatment of humans.

14. Use of an oral preparation that contains a glucosamine, derivatives thereof and/or salts thereof in combination with the preparation according to claim 1 for the treatment of nail, cartilage, bone, joint, muscle and tendon disorders.

15. Use according to claim 14, wherein the oral preparation contains N-acetyl-Dglucosamine or a pharmaceutically acceptable salt thereof, preferably N-acetyl-Dglucosamine HCl.

16. Kit comprising an oral preparation that contains a glucosamine and a topical preparation that contains polydimethylsiloxane as a combination for simultaneous, separate or successive administration.

17. Kit according to claim 16, for the treatment of nail, cartilage, bone, joint, muscle and tendon disorders.

18. Use of polydimethylsiloxane having a viscosity of 10,000 to 100,000 cSt as carrier in a topical preparation, wherein the preparation contains at least 50% (m/m), based on the weight of the preparation, of polydimethylsiloxane.

19. Use according to claim 18, wherein the topical preparation is used for the external treatment of nails, cartilage, bones, joints, muscles and tendons.

20. Use according to claim 18, wherein the topical preparation contains at least 50% (m/m) polydimethylsiloxane.
